(19) United States
(12) Patent Application Publication
(10) Pub. No.: US 2026/0188495 A1
(43) Pub. Date: Jul. 2, 2026

(54) AI CLINICAL DECISION SUPPORT SYSTEM USING CONNECTIVITY MODEL ANALYSIS

(71) Applicant: UNIVERSITY OF SHARJAH, SHARJAH (AE)

(72) Inventors: Saddaf RUBAB, Sharjah (AE); Munsif Ali JATOI, Sharjah (AE)

(21) Appl. No.: 19/430,741

(22) Filed: Dec. 23, 2025

Related U.S. Application Data
(60) Provisional application No. 63/740,621, filed on Dec. 31, 2024.

(51) Int. Cl. G16H 50/20; G16H 10/60; G16H 15/00; G16H 20/00; G16H 30/40

(52) U.S. Cl. CPC G16H 50/20; G16H 10/60; G16H 15/00; G16H 20/00; G16H 30/40

(57) ABSTRACT

The present disclosure provides an AI-based clinical decision support system comprising an input module configured to receive clinical information comprising brain scan data, an analysis module configured to parse the clinical information using statistical measures from functional connectivity analysis with counterfactual explanations to identify brain connectivity patterns associated with brain disorders, and an output module configured to present a recommended diagnosis and explanation comprising attribution information identifying connectivity features contributing to the diagnosis. The brain scan data comprises functional magnetic resonance imaging, electroencephalography, and magnetoencephalography data. The statistical measures comprise functional connectivity analysis and graph theory metrics including degree metrics, betweenness centrality measures, and clustering coefficients. The analysis module comprises a functional connectivity engine configured to process brain scan data and generate connectivity features, a feature bank configured to store connectivity features, and modeling backbones configured to analyze connectivity features using machine learning techniques.

### FIELD OF INVENTION

The present disclosure relates to artificial intelligence systems for medical applications, and more particularly to an artificial intelligence-based clinical decision support system that uses functional connectivity model analysis for diagnosing brain disorders.

### BACKGROUND

Artificial intelligence has experienced substantial advancement across numerous fields, leading to reduced human workload and enhanced quality of life through modern computational approaches and tools. The integration of AI technologies into healthcare applications has shown particular promise, with potential applications spanning patient triage, diagnostic assistance, and clinical decision-making processes.

Clinical decision support systems represent a growing area of interest within healthcare technology, offering computerized tools to assist medical practitioners in making informed decisions. These systems can analyze complex medical data and provide recommendations to support clinical workflows. However, the implementation of AI-driven clinical decision support faces several challenges that limit widespread adoption in medical settings.

One challenge relates to the transparency of AI models used in clinical applications. Many high-performance AI systems operate as “black boxes,” where the reasoning behind their outputs remains opaque to users. This lack of transparency can create difficulties for medical practitioners who need to understand the basis for AI-generated recommendations. The ability to explain and interpret AI decisions becomes particularly relevant in healthcare contexts where practitioners must justify their clinical choices.

Brain-related medical conditions represent a substantial healthcare burden, involving considerable economic costs and affecting millions of individuals. The diagnosis and treatment of neurological and psychiatric disorders often require specialized expertise and can involve complex decision-making processes. Traditional diagnostic approaches benefit from computational support that can analyze brain imaging data and other clinical information.

Functional connectivity analysis has emerged as a technique for examining relationships between different brain regions using neuroimaging data. This approach can provide insights into brain network organization and has potential applications in understanding various neurological and psychiatric conditions. The integration of such analytical methods with clinical decision support systems could offer new approaches to brain disorder diagnosis and treatment planning.

The development of explainable AI methods addresses the transparency challenges associated with complex AI models. These approaches aim to provide interpretable explanations for AI-generated outputs, enabling users to understand the reasoning behind automated decisions. The application of explainable AI techniques to clinical decision support could help bridge the gap between AI capabilities and clinical practice requirements.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, an explainable AI-based clinical decision support system is provided. The system comprises an input module configured to receive clinical information. The system comprises an analysis module configured to parse the clinical information using a combination of statistical measures obtained through functional connectivity with reasoning through counterfactual explanations. The system comprises an output module configured to present to a user one or more of a recommended diagnosis or a recommended decision, and an explanation for either the recommended diagnosis or recommended decision.

According to other aspects of the present disclosure, the system includes one or more of the following features. The clinical information is a brain scan. The statistical measures comprise functional connectivity analysis and graph theory.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a flowchart of an explainable AI-based clinical decision support system, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to an artificial intelligence (AI)-based clinical decision support system (CDSS) configured to assist medical personnel in diagnosing brain disorders. The system is specifically designed for diagnosing brain disorders such as stress, depression, anxiety, and epilepsy. The CDSS provides enhanced decision confidence through transparent and understandable AI-driven analysis, addressing challenges associated with the lack of transparency in conventional AI models used in medical applications. The proposed explainable AI-based clinical decision support system comprises three main components that work together to provide comprehensive diagnostic support. An input module is configured to receive clinical information from various sources. In some cases, the clinical information includes brain scan data or other neurological data relevant to brain disorder diagnosis. The input module serves as the primary interface for data acquisition in the diagnostic process.

An analysis module is configured to parse the clinical information using a combination of statistical measures obtained through functional connectivity with reasoning through counterfactual explanations. The analysis module employs advanced computational techniques to process and analyze the received clinical information. In some cases, the statistical measures comprise functional connectivity analysis and graph theory. The analysis module utilizes these statistical approaches to identify patterns and relationships within the clinical data that may be indicative of specific brain disorders. An output module is configured to present to a user one or more of a recommended diagnosis or a recommended decision, and an explanation for either the recommended diagnosis or recommended decision. The output module provides medical personnel with actionable insights derived from the analysis performed by the analysis module. In some cases, the explanations provided by the output module enhance the transparency and understandability of the AI-driven diagnostic process, enabling medical personnel to make more informed decisions regarding patient care.

The proposed system represents an interdisciplinary approach that combines advanced statistical methods, computer science, and neurosciences to create a comprehensive diagnostic tool. The integration of these diverse fields enables the system to leverage mathematical and statistical knowledge, including functional connectivity analysis and graph theory, in conjunction with explainable AI techniques. This multidisciplinary foundation provides the system with the capability to generate explanations for automated decision-making processes, thereby creating a transparent and informed solution for brain disorder diagnosis.

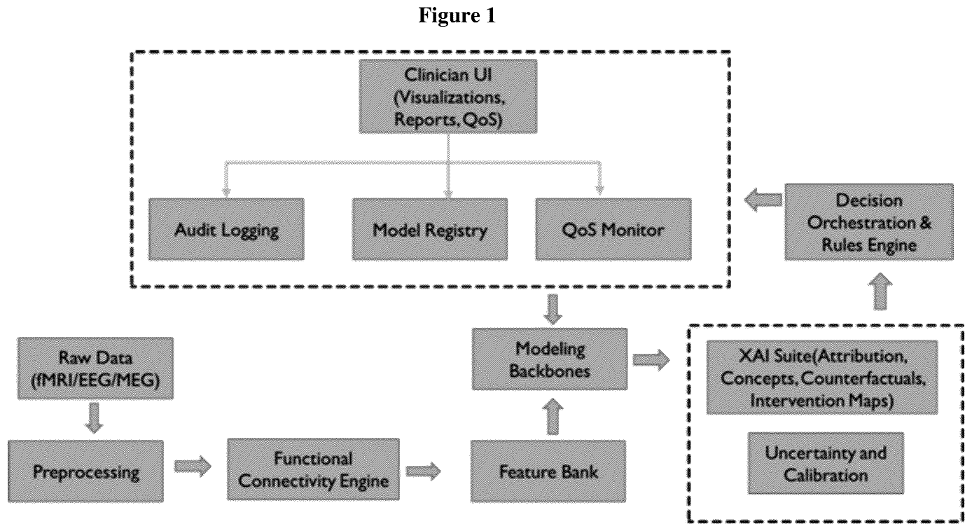

Referring to FIG. 1, the system architecture demonstrates a comprehensive workflow for processing brain data through multiple stages to generate explainable clinical decisions. The architecture illustrates the flow of information from raw brain data acquisition through various processing components to produce final clinical outputs with associated explanations. The system development process follows four major steps as shown in FIG. 1. The first step involves collection of brain data from multiple neuroimaging modalities. Raw data is acquired from functional magnetic resonance imaging (fMRI), electroencephalography (EEG), and magnetoencephalography (MEG) sources. These brain imaging modalities provide different types of neurological information that can be processed by the system for diagnostic purposes.

The second step involves developing a functional connectivity model based on the collected brain data. As shown in FIG. 1, the raw data undergoes preprocessing before being processed by a functional connectivity engine. The functional connectivity engine analyzes the relationships and connections between different brain regions to create a comprehensive connectivity model. The processed connectivity information is then directed to a feature bank that stores relevant features extracted from the connectivity analysis. With continued reference to FIG. 1, the third step involves providing explanations to the model for explainability and further diagnoses. The system incorporates an XAI Suite that encompasses multiple explainability components including attribution, concepts, counterfactuals, and intervention maps. The XAI Suite also includes uncertainty and calibration components that contribute to the overall explainability framework. These components work together to generate explanations for the model's decision-making processes.

The fourth step involves developing the CDSS based on the XAI functional connectivity model. As further shown in FIG. 1, modeling backbones receive input from the feature bank and interact with the XAI Suite to provide explainable outputs. A decision orchestration and rules engine receive input from the XAI Suite and generate synthesized outputs that support clinical decision-making. The system incorporates coupling of causality and explainability to provide improved reasoning chains for patients. The integration of causality analysis with explainability mechanisms enable the system to not only identify potential diagnoses but also provide clear reasoning for how specific conclusions were reached. This coupling is facilitated through the interaction between the modeling backbones and the XAI Suite, where causal relationships identified in the functional connectivity model is combined with explainable AI techniques to generate comprehensive reasoning chains.

The final clinical output is presented through a clinician user interface that provides visualizations, reports, and quality of service monitoring. As shown in FIG. 1, the interface includes audit logging, model registry, and QoS monitor components that work together to provide transparency and traceability in the clinical decision support process. This comprehensive interface enables medical personnel to access both the diagnostic recommendations and the underlying explanations that support those recommendations. The input module serves as the primary data acquisition component of the explainable AI-based clinical decision support system, configured to receive and process clinical information from multiple neuroimaging sources. The input module is designed to handle diverse types of brain scan data, enabling comprehensive analysis of neurological conditions through multiple data acquisition modalities. In some cases, the input module provides standardized interfaces for receiving clinical information from various hospital and clinical systems.

Referring to FIG. 1, the input module receives raw data from three primary neuroimaging modalities: functional magnetic resonance (fMRI), electroencephalography (EEG), and magnetoencephalography (MEG). Each of these modalities provide different types of neurological information that contribute to the overall diagnostic capability of the system. The fMRI data provides information about blood flow and oxygenation changes in brain regions, while EEG data captures electrical activity patterns across the brain. MEG data offers complementary information about magnetic fields generated by neural activity. The data collection methods implemented by the input module accommodates the varying formats and specifications associated with different neuroimaging equipment and protocols. In some cases, the input module includes format conversion capabilities to standardize data from different sources into a common format suitable for subsequent processing. The input module also incorporates data validation mechanisms to ensure that received clinical information meets quality standards for reliable analysis.

With continued reference to FIG. 1, the raw data received by the input module undergoes preprocessing steps before being processed by the functional connectivity engine. The preprocessing stage is configured to address various data quality issues that can affect the accuracy of subsequent analysis. These preprocessing steps include noise removal techniques specifically designed for each type of neuroimaging data, as different modalities may be subject to different types of noise and artifacts. The noise removal techniques applied during preprocessing include filtering methods to eliminate high-frequency noise, motion correction algorithms to address patient movement artifacts, and baseline correction procedures to normalize signal variations. In some cases, the preprocessing also includes temporal filtering to remove physiological noise such as cardiac and respiratory artifacts that can interfere with brain signal analysis. These preprocessing steps are automatically applied by the input module to ensure consistent data quality across different data sources.

In an embodiment, data reduction techniques are also implemented during the preprocessing stage to manage the large volumes of data typically generated by neuroimaging modalities. As further shown in FIG. 1, the preprocessing component applies dimensionality reduction methods to extract relevant features while reducing computational complexity for subsequent analysis stages. These reduction techniques include principal component analysis or other statistical methods that preserve important signal characteristics while eliminating redundant information. The input module incorporates quality assessment mechanisms to evaluate the suitability of received clinical information for diagnostic analysis. These quality assessment procedures include signal-to-noise ratio calculations, artifact detection algorithms, and completeness checks to ensure that sufficient data is available for reliable connectivity analysis. In some cases, the input module provides feedback to users regarding data quality issues that affect the reliability of diagnostic outputs.

The preprocessed data is then formatted and prepared for transmission to the functional connectivity engine, as shown in FIG. 1. The input module ensures that the processed clinical information maintains appropriate metadata and temporal relationships necessary for functional connectivity analysis. This preparation includes synchronization of data from different modalities when multiple types of neuroimaging data are available for the same patient, enabling comprehensive multi-modal analysis of brain connectivity patterns.

The functional connectivity engine serves as a central processing component that transforms preprocessed brain data into statistical representations suitable for diagnostic analysis. As shown in FIG. 1, the functional connectivity engine receives preprocessed data from the input module and applies various computational techniques to identify and quantify relationships between different brain regions. The functional connectivity engine is configured to process data from multiple neuroimaging modalities simultaneously, enabling comprehensive analysis of brain connectivity patterns across different measurement domains. The functional connectivity engine employs statistical measures to convert raw neuroimaging signals into meaningful connectivity representations. These statistical measures include fundamental statistical calculations such as mean, variance, covariance, and correlation coefficients that characterize the relationships between brain region activities. The mean values provide baseline activity levels for different brain regions, while variance calculations indicate the degree of activity fluctuation within each region over time.

Covariance analysis performed by the functional connectivity engine reveals how brain regions vary together, indicating potential functional relationships between different areas of the brain. Correlation coefficients are calculated to quantify the strength and direction of linear relationships between brain region activities. In some cases, these correlation measures are computed across different time windows to capture both static and dynamic connectivity patterns that are relevant for diagnosing various brain disorders. With continued reference to FIG. 1, the functional connectivity engine incorporates graph theory metrics to provide additional analytical perspectives on brain connectivity patterns. Graph theory analysis treats brain regions as nodes and connectivity relationships as edges, enabling the application of network analysis techniques to understand brain organization and function. The functional connectivity engine calculates various graph theory metrics that characterize different aspects of brain network topology.

As further shown in FIG. 1, effective connectivity analysis utilizes time-series analysis methods to identify temporal precedence relationships between brain region activities. These methods include Granger causality analysis, dynamic causal modeling, or other techniques that can distinguish between regions that drive activity changes and regions that respond to those changes. The effective connectivity analysis provides additional explanatory power for understanding the mechanisms underlying brain disorders and the rationale for diagnostic recommendations. The statistical representations generated by the functional connectivity engine are systematically organized and stored in a feature bank for subsequent modeling processes. As shown in FIG. 1, the feature bank serves as a repository for the various connectivity measures, graph theory metrics, and effective connectivity parameters calculated by the functional connectivity engine. The feature bank organizes these features in a structured format that facilitates efficient access by downstream modeling components.

In an embodiment, decision tree models are incorporated within the modeling backbones to provide rule-based diagnostic pathways that can be easily understood and validated by medical professionals. The decision trees create hierarchical decision structures that partition the feature space based on connectivity thresholds, generating clear decision rules that can be followed step-by-step. These tree-based models provide natural explanations for diagnostic decisions by showing the sequence of connectivity feature evaluations that lead to specific diagnostic conclusions. With continued reference to FIG. 1, the decision tree approach enables the modeling backbones to generate diagnostic pathways that mirror clinical reasoning processes used by medical professionals. The tree structures identify the most discriminative connectivity features at each decision point, providing insights into which aspects of brain connectivity are most relevant for distinguishing between different diagnostic categories. This hierarchical approach facilitates clinical validation of the diagnostic process by enabling medical experts to evaluate the logical consistency of the decision pathways.

K-nearest neighbors (KNN) algorithms are implemented within the modeling backbones to provide instance-based learning capabilities that compare new patient cases to similar cases in the training dataset. The KNN approach identifies the most similar historical cases based on connectivity feature similarity and use the diagnoses of these similar cases to inform diagnostic recommendations for new patients. This similarity-based approach provides intuitive explanations by showing medical personnel which previous cases are most similar to the current patient. As shown in FIG. 1, the modeling backbones interact with the XAI Suite to enhance the explainability of the analytical processes and to generate comprehensive explanations for diagnostic decisions. This interaction enables the integration of model-specific explanations with broader explainability frameworks, creating multi-layered explanations that address different aspects of the diagnostic process. The modeling backbones provide feature importance scores, decision pathways, and similarity assessments that are then processed by the XAI Suite to generate user-friendly explanations for medical personnel.

The XAI Suite serves as a comprehensive explainability framework that enhances the transparency and interpretability of the diagnostic decisions generated by the modeling backbones. As shown in FIG. 1, the XAI Suite encompasses multiple explainability components that work together to provide multi-faceted explanations for clinical decision support. The XAI Suite is configured to receive analytical outputs from the modeling backbones and transform these outputs into understandable explanations that can be effectively utilized by medical personnel in clinical settings. The attribution component of the XAI Suite is configured to identify and quantify the contribution of individual connectivity features to specific diagnostic decisions. Attribution analysis determines which brain connectivity patterns have the greatest influence on diagnostic recommendations, enabling medical personnel to understand the neurological basis for AI-generated conclusions. In some cases, the attribution component generates feature importance scores that rank connectivity measures according to their diagnostic relevance for specific brain disorders.

The XAI Suite implements model-agnostic explainability techniques that can provide explanations for diagnostic decisions regardless of the specific machine learning algorithms used by the modeling backbones. The XAI Suite also implements model-specific explainability techniques that leverage the internal structure and parameters of specific machine learning algorithms used by the modeling backbones. For linear models such as linear regression and logistic regression, the XAI Suite extracts and interprets model coefficients to provide direct explanations of feature importance and effect directions. These coefficient-based explanations show medical personnel how changes in connectivity features translate to changes in diagnostic probabilities. With continued reference to FIG. 1, transparency is achieved through comprehensive documentation and communication of the analytical processes used throughout the diagnostic pipeline. The XAI Suite maintains detailed records of the explainability techniques applied, the parameters used for explanation generation, and the assumptions underlying different explanation types. This documentation enables medical personnel to understand not only the diagnostic recommendations but also the methods used to generate explanations for those recommendations.

The decision orchestration and rules engine serve as a central coordination component that synthesizes the analytical outputs from the XAI Suite to generate comprehensive clinical recommendations. As shown in FIG. 1, the decision orchestration and rules engine receive input from the XAI Suite and process this information through systematic decision-making frameworks to produce actionable clinical outputs. The decision orchestration and rules engine is configured to integrate multiple types of explanatory information, including attribution scores, conceptual insights, counterfactual analyses, and uncertainty estimates, into coherent diagnostic recommendations that can be effectively utilized by medical personnel. The synthesis process implemented by the decision orchestration and rules engine involves the application of clinical decision rules that encode medical knowledge and best practices for brain disorder diagnosis. These decision rules may be developed in collaboration with medical experts and incorporate established diagnostic criteria for various neurological conditions. The rules engine evaluates the connectivity-based evidence provided by the XAI Suite against these clinical criteria to determine the most appropriate diagnostic recommendations for individual patient cases.

As further shown in FIG. 1, the decision orchestration and rules engine generate multiple types of clinical outputs that address different aspects of diagnostic decision-making. Diagnostic recommendations are produced that identify the most likely brain disorders based on the connectivity analysis and provide confidence estimates for these diagnoses. Treatment decision support also may be generated that suggests appropriate therapeutic approaches based on the identified connectivity patterns and their relationship to established treatment protocols. Integration with clinical guidelines and protocols are implemented within the rules engine to ensure that diagnostic recommendations align with established medical standards and institutional practices. The rules engine references current diagnostic criteria for brain disorders and incorporates updates to clinical guidelines as they become available. This integration helps to ensure that the AI-generated recommendations complement rather than conflict with standard clinical practices and facilitate the adoption of the system within existing healthcare workflows. The decision orchestration and rules engine implement adaptive learning capabilities that enable the system to refine decision rules based on clinical feedback and outcomes data. When medical personnel provide feedback about the accuracy or clinical utility of diagnostic recommendations, this information may be used to adjust decision thresholds, modify rule priorities, or update consensus mechanisms. This adaptive capability enables the system to continuously improve its clinical performance and to better align with the needs and preferences of medical users.

As further shown in FIG. 1, the QoS monitor component implements dashboard interfaces that provide visual summaries of system performance metrics and trends over time. These dashboards display key performance indicators such as diagnostic throughput, average confidence levels, and user engagement statistics that help clinical administrators evaluate the effectiveness of the system within their healthcare environment. Alert mechanisms may be implemented to notify administrators when performance metrics fall below acceptable thresholds or when system issues require attention. The audit logging component provides comprehensive documentation of all diagnostic processes, user interactions, and system decisions to ensure transparency and traceability throughout the clinical decision support workflow. Audit logs record detailed information about the data inputs used for each diagnostic case, the analytical methods applied, the explanatory techniques employed, and the final recommendations generated. This comprehensive logging supports quality assurance processes, regulatory compliance requirements, and clinical research activities that rely on detailed documentation of AI-assisted diagnostic procedures.

With continued reference to FIG. 1, the model registry component maintains comprehensive records of the AI models, algorithms, and configurations used for diagnostic analysis, enabling version control and reproducibility of analytical results. The model registry tracks different versions of machine learning models as they are updated and refined, ensuring that diagnostic recommendations can be traced to specific model configurations and training datasets. This version control capability is particularly important for maintaining consistency in clinical applications and for supporting regulatory validation of AI-based diagnostic tools. The output module serves as the primary interface component responsible for presenting recommended diagnoses or decisions along with corresponding explanations to medical users in accessible and clinically relevant formats. The output module receives synthesized recommendations from the decision orchestration and rules engine and formats this information for effective presentation through the clinician user interface. The formatting process organizes diagnostic information according to clinical priorities and adapts the presentation style to match the preferences and workflow requirements of different types of medical users.

As shown in FIG. 1, the output module implements adaptive presentation mechanisms that customize the format and content of diagnostic information based on user roles, clinical context, and institutional preferences. Different presentation modes are available for different types of medical personnel, such as neurologists, psychiatrists, or primary care physicians, with each mode emphasizing the aspects of diagnostic information most relevant to specific clinical specialties. The adaptive presentation helps to ensure that AI-generated insights are communicated in ways that align with the knowledge base and decision-making processes of different medical professionals. Integration with clinical workflow systems is implemented within the output module to ensure that diagnostic recommendations and explanations are delivered at appropriate points in the clinical decision-making process. The output module coordinates with electronic health record systems, clinical decision support platforms, and other healthcare information systems to provide seamless integration of AI-generated insights with existing clinical workflows. This integration helps to minimize disruption to established clinical practices while maximizing the utility of connectivity-based diagnostic information.

The output module also implements feedback collection mechanisms that enable medical personnel to provide input about the accuracy, relevance, and clinical utility of diagnostic recommendations and explanations. This feedback is used to validate system performance, identify areas for improvement, and support continuous learning processes that enhance the clinical effectiveness of the AI-based diagnostic support system. The feedback collection includes structured rating systems, free-text comments, and outcome tracking that provide comprehensive assessment of clinical impact and user satisfaction.

With continued reference to FIG. 1, the comprehensive integration of visualizations, reports, and quality of service monitoring within the clinician user interface creates a unified platform that supports all aspects of AI-assisted clinical decision-making for brain disorder diagnosis. The coordinated presentation of diagnostic recommendations, explanatory insights, performance metrics, and audit information enables medical personnel to develop confidence in AI-generated recommendations while maintaining appropriate clinical oversight and professional judgment. This integrated approach facilitates the effective adoption of explainable AI technology within clinical practice while ensuring that the benefits of advanced connectivity analysis are accessible to medical professionals across different healthcare settings. The explainable AI-based clinical decision support system is configured for versatile application across multiple types of healthcare and research environments, enabling widespread deployment of connectivity-based diagnostic capabilities. The system architecture is designed to accommodate the diverse operational requirements, technical infrastructures, and clinical workflows found in different institutional settings. This versatility enables healthcare organizations and research institutions to leverage advanced brain connectivity analysis regardless of their specific operational context or technical capabilities.

In another embodiment, the system's implementation in XAI research centers facilitates interdisciplinary collaboration between computer scientists, neuroscientists, and medical professionals working to advance the field of explainable medical AI. Research applications focus on optimizing the balance between diagnostic accuracy and explanation quality, developing new methods for uncertainty quantification in medical AI, and investigating how different types of explanations affect clinical decision-making processes. These research activities contribute to the broader advancement of trustworthy AI in healthcare applications. Clinical deployment environments include outpatient neurology clinics, psychiatric practices, and specialized brain disorder treatment centers that require diagnostic support for ongoing patient care. Clinic-based implementations emphasize the system's ability to support longitudinal patient monitoring, treatment response assessment, and diagnostic refinement over extended periods of care. The explainable interface enables clinic-based physicians to track changes in brain connectivity patterns over time and to understand how these changes relate to treatment outcomes and disease progression.

In another embodiment, the proposed system is designed with portable features that enable deployment across different clinical environments without requiring extensive infrastructure modifications or specialized technical support. Portable design characteristics include compatibility with standard computing hardware commonly available in healthcare settings, minimal requirements for specialized software installations, and flexible data input mechanisms that can accommodate different neuroimaging equipment and data formats. These portable features reduce the barriers to system adoption and enable rapid deployment in diverse clinical contexts. Cloud-based deployment options enhance the portability of the system by enabling access to diagnostic capabilities through standard web browsers or lightweight client applications. Cloud implementations provide scalable computational resources that can accommodate varying diagnostic volumes across different institutions, while maintaining consistent analytical capabilities and explanation quality. The cloud-based approach also facilitates system updates and maintenance without requiring local technical expertise, reducing the operational burden on healthcare institutions.

The proposed explainable AI-based clinical decision support system provides substantial cost-effectiveness benefits for healthcare institutions through multiple mechanisms that reduce operational expenses while maintaining or improving diagnostic quality. The automated analysis capabilities eliminate the need for extensive manual review of brain connectivity data, reducing the personnel time required for diagnostic assessments. Healthcare institutions realize cost savings through reduced labor costs associated with diagnostic workups, as the system performs complex connectivity analyses that would otherwise require specialized neuroimaging expertise and extended analysis time.

Diagnostic efficiency improvements contribute to cost-effectiveness by enabling healthcare institutions to process larger numbers of patients with existing resources. The rapid analysis capabilities reduce patient throughput times while providing comprehensive diagnostic assessments, enabling more efficient utilization of imaging equipment and clinical personnel. Healthcare institutions benefit from increased diagnostic capacity without proportional increases in staffing or equipment costs, improving the overall economic efficiency of brain disorder diagnostic services. The system minimizes risk of diagnostic errors through the application of standardized analytical procedures that reduce variability in diagnostic quality across different practitioners and clinical settings. Human diagnostic errors result from fatigue, inexperience, or cognitive biases that can affect the interpretation of complex brain imaging data. The automated connectivity analysis provides consistent, objective assessments that complement human clinical judgment while reducing the likelihood of diagnostic oversights or misinterpretations that can lead to inappropriate treatments and increased healthcare costs.

Time-efficiency benefits are achieved through the rapid processing of brain connectivity data that would otherwise require extensive manual analysis by specialized personnel. Traditional connectivity analysis requires hours or days of expert review to identify relevant patterns and generate diagnostic insights. The automated analysis capabilities provide comprehensive connectivity assessments within minutes of data acquisition, enabling real-time diagnostic support that can accelerate clinical decision-making processes and reduce patient waiting times. Reduced human labor requirements result from the automation of complex analytical tasks that traditionally require specialized expertise in neuroimaging analysis and brain connectivity interpretation. The system enables general practitioners and non-specialist physicians to access advanced brain connectivity insights without requiring extensive training in neuroimaging analysis techniques. This democratization of advanced diagnostic capabilities reduces the dependence on scarce specialist resources while maintaining high standards of diagnostic quality.

The transparent AI approach builds trust between medical personnel and AI-assisted diagnostic tools by providing clear explanations of analytical methods and diagnostic reasoning. The comprehensive benefits achieved through cost-effectiveness, time-efficiency, error reduction, and quality enhancement position the explainable AI-based clinical decision support system as a valuable tool for improving healthcare delivery while managing healthcare costs. Healthcare institutions achieve improved patient outcomes while maintaining or reducing operational expenses, supporting sustainable healthcare delivery models that can accommodate growing demand for brain disorder diagnostic services. The combination of advanced analytical capabilities with transparent explanatory mechanisms enables healthcare providers to leverage artificial intelligence benefits while maintaining the human-centered approach that is fundamental to effective medical care.

A number of implementations have been described. Nevertheless, it will be understood that various modifications may be made without departing from the spirit and scope of the disclosure. Accordingly, other implementations are within the scope of the following claims.

1. An AI-based clinical decision support system, comprising:
an input module configured to receive clinical information comprising brain scan data;
an analysis module configured to parse the clinical information using a combination of statistical measures obtained through functional connectivity analysis with reasoning through counterfactual explanations to identify brain connectivity patterns associated with brain disorders; and
an output module configured to present to a user a recommended diagnosis for a brain disorder and an explanation for the recommended diagnosis, wherein the explanation comprises attribution information identifying connectivity features that contribute to the recommended diagnosis.

2. The system of claim 1, wherein the brain scan data comprises data from at least one of functional magnetic resonance imaging (fMRI), electroencephalography (EEG), and magnetoencephalography (MEG).

3. The system of claim 1, wherein the statistical measures comprise functional connectivity analysis and graph theory metrics.

4. The system of claim 3, wherein the graph theory metrics comprise degree metrics, betweenness centrality measures, and clustering coefficients.

5. The system of claim 1, wherein the analysis module comprises:
a functional connectivity engine configured to process the brain scan data and generate connectivity features;
a feature bank configured to store the connectivity features; and
modeling backbones configured to analyze the connectivity features using machine learning techniques.

6. The system of claim 5, wherein the modeling backbones comprise at least one of linear regression models, logistic regression models, decision tree models, and k-nearest neighbors algorithms.

7. The system of claim 1, wherein the output module comprises a clinician user interface configured to present visualizations of brain connectivity patterns and generate clinical reports summarizing the recommended diagnosis and explanation.

8. A method for providing explainable clinical decision support for brain disorder diagnosis, comprising:
receiving clinical information comprising brain scan data from at least one neuroimaging modality;
processing the clinical information through a functional connectivity engine to generate statistical measures comprising functional connectivity analysis and graph theory metrics;
applying explainable artificial intelligence techniques including counterfactual explanations to the statistical measures to generate diagnostic insights; and
presenting a recommended diagnosis for a brain disorder along with an explanation that identifies brain connectivity patterns that contribute to the recommended diagnosis.

9. The method of claim 8, wherein the at least one neuroimaging modality comprises functional magnetic resonance imaging (fMRI), electroencephalography (EEG), or magnetoencephalography (MEG).

10. The method of claim 8, wherein the graph theory metrics comprise degree metrics, betweenness centrality measures, and clustering coefficients.

11. The method of claim 8, wherein applying explainable artificial intelligence techniques further comprises generating attribution information that identifies connectivity features contributing to the recommended diagnosis.

12. The method of claim 11, wherein the explainable artificial intelligence techniques further comprise generating intervention maps that illustrate potential effects of therapeutic interventions on brain connectivity patterns.

13. The method of claim 8, further comprising a step of preprocessing the brain scan data to remove noise and artifacts before processing through the functional connectivity engine.

14. The method of claim 13, wherein the step of preprocessing comprises applying temporal filtering to remove physiological noise and motion correction algorithms to address patient movement artifacts.

15. An artificial intelligence system for brain disorder diagnosis, comprising:
a functional connectivity engine configured to process brain scan data and generate connectivity features using statistical measures and graph theory analysis;
an XAI suite comprising attribution, counterfactuals, and uncertainty components configured to generate explanations for diagnostic decisions based on the connectivity features; and
a decision orchestration engine configured to synthesize outputs from the XAI suite to generate a recommended diagnosis for a brain disorder with associated explanatory information that enables medical personnel to understand the neurobiological basis for the diagnostic recommendation.

16. The system of claim 15, wherein the brain scan data comprises data from at least one of functional magnetic resonance imaging (fMRI), electroencephalography (EEG), and magnetoencephalography (MEG).

17. The system of claim 15, wherein the statistical measures comprise correlation coefficients, covariance analysis, and mean activity levels for different brain regions.

18. The system of claim 17, wherein the graph theory analysis comprises degree metrics, betweenness centrality measures, and clustering coefficients that characterize brain network topology.

19. The system of claim 15, wherein the XAI suite further comprises intervention maps configured to illustrate potential effects of therapeutic interventions on brain connectivity patterns.

20. The system of claim 19, wherein the decision orchestration engine is configured to generate clinical reports that include visualizations of brain connectivity networks and confidence estimates for the recommended diagnosis.
